(19) United States
(12) Patent Application Publication
(10) Pub. No.: US 2014/0322712 A1
(43) Pub. Date: Oct. 30, 2014

(54) DIAGNOSIS AND TREATMENT OF MACULAR DEGENERATION

(71) Applicant: THE REGENTS OF THE UNIVERSITY OF MICHIGAN, Ann Arbor, MI (US)

(72) Inventor: Goncalo Abecasis, Ann Arbor, MI (US)

(73) Assignee: THE REGENTS OF THE UNIVERSITY OF MICHIGAN, Ann Arbor, MI (US)

(21) Appl. No.: 14/264,799

(22) Filed: Apr. 29, 2014

Related U.S. Application Data
(60) Provisional application No. 61/817,167, filed on Apr. 29, 2013.

(51) Int. Cl. C12Q 1/68

(52) U.S. Cl. CPC C12Q 1/6883
USPC 435/6.11

(57) ABSTRACT

The present invention relates generally to biomarkers for macular degeneration. In particular, the biomarkers and related compositions and methods of the present invention find use in diagnostic, therapeutic, research, and drug screening applications.

### CROSS-REFERENCE TO RELATED APPLICATION

The present application claims priority to U.S. Provisional Patent Application Ser. No. 61/817,167 filed Apr. 29, 2013, which is hereby incorporated by reference in its entirety.

### FIELD OF THE INVENTION

The present invention relates generally to biomarkers for macular degeneration. In particular, the biomarkers and related compositions and methods of the present invention find use in diagnostic, therapeutic, research, and drug screening applications.

### BACKGROUND OF THE INVENTION

Genetic and environmental factors contribute to age-related macular degeneration (AMD), a major cause of vision loss in elderly individuals (Priya et al. Ophthalmology 119, 2526-36 (2012); Swaroop et al Annu Rev Genomics Hum Genet 10, 19-43 (2009); Friedman et al. Archives of Ophthalmology 122, 564-72 (2004); herein incorporated by reference in their entireties). The pioneering discovery of association of AMD with complement factor H (CFH4-6) was quickly followed by the identification of additional susceptibility loci that now include ARMS2/HTRA17 and complement genes C3, C2/CFB and CFI8-11. Genome-wide association studies (GWAS) of AMD cases and controls have now revealed common susceptibility variants at ˜20 different loci and begun to uncover specific cellular pathways involved in AMD biology (Fritsche et al. Nature Genetics (2013); Arakawa et al. Nat Genet 43, 1001-4 (2011); herein incorporated by reference in their entireties). While common variants tag the associated genomic region, rare variants may provide more specific clues about the underlying disease mechanism (Nejentsev et al. Science 324, 387-9 (2009); herein incorporated by reference in its entirety). A need exists to identify such rare variants.

### SUMMARY

In some embodiments, the present invention provides a method for characterizing a subject's risk for developing age-related macular degeneration (AMD) comprising detecting the presence of or the absence of one or more polymorphisms on or near the genes CFH, ARMS2/HTRA1, C2/CFB, C3, CFI, SYN3/TIMP3, and LIPC. In some embodiments, one or more polymorphisms are in or near CFH and/or C3. In some embodiments, the polymorphisms are one or more of rs147859257 (e.g., G allele), rs2230199 (e.g., C allele), and/or rs121913059 (e.g., T allele), alone, or in combination with other markers (e.g. known markers), or alternatively detection of polymorphisms or sequences in linkage disequilibrium with any of the above markers. In some embodiments, biomarkers comprise polymorphisms that result in C3 variant K155Q (or a variant comprising the K155Q mutation), C3 variant R102G (or a variant comprising the R102G mutation), and/or CFH variant R1210C (or a variant comprising the R1210C mutation). In some embodiments, variants disrupt the C3/CFH interaction. In some embodiments, polymorphisms result in amino acids substitutions at the C3/CFH interface. In some embodiments, the method comprises detecting the presence of or the absence of two or more polymorphisms. In some embodiments, the method comprises detecting the presence of or the absence of five or more polymorphisms. In some embodiments, the method comprises detecting the presence of or the absence of seven or more polymorphisms. In some embodiments, the method comprises detecting the presence of or the absence of nine polymorphisms. In some embodiments, detection of the one or more polymorphisms (e.g. 1, 2, 3, 4, 5, 6, 7, 8, 9, 10, >10) indicates an elevated risk of developing AMD.

In some embodiments, in addition to one or more of rs147859257 (e.g., G allele), rs2230199 (e.g., C allele), and/or rs121913059 (e.g., T allele), the present invention comprises detection of the presence of one or more of rs10737680, rs3793917, rs429608, rs2230199, rs2285714, rs1329424, rs9380272, rs9621532, and rs493258 (See U.S. Pat No. 8,119,348; herein incorporated by reference in its entirety) and/or one or more of rs2274700, rs1410996, rs7535263, rs10801559, rs3766405, rs10754199, rs1329428, rs10922104, rs1887973, rs10922105, rs4658046, rs10465586, rs3753395, rs402056, rs7529589, rs7514261, rs10922102, rs10922103, rs800290, rs1061147, rs1061170, rs1048663, rs412852, rs11582939, and rs1280514 (U.S. Pub. No. 2009/0203001; herein incorporated by reference in its entirety).

In some embodiments, the present invention provides a panel of AMD markers. In some embodiments, the present invention comprises a panel of two or more AMD markers. In some embodiments, the present invention comprises a panel of three or more AMD markers. In some embodiments, the present invention comprises a panel of four or more AMD markers. In some embodiments, the present invention comprises a panel of five or more AMD markers. In some embodiments, the present invention comprises a panel of six or more AMD markers. In some embodiments, the present invention comprises a panel of seven or more AMD markers. In some embodiments, the present invention comprises a panel of eight or more AMD markers. In some embodiments, the present invention comprises a panel of nine or more AMD markers.

In some embodiments, the present invention comprises a panel of markers comprising the AMD marker rs147859257 (e.g., G allele) any combination of AMD markers disclosed herein or elsewhere. In some embodiments, the present invention comprises a panel of markers comprising the AMD marker rs2230199 (e.g., C allele) and any combination of AMD markers disclosed herein or elsewhere. In some embodiments, the present invention comprises a panel of markers comprising the AMD marker rs121913059 (e.g., T allele) and any combination of AMD markers disclosed herein or elsewhere.

In some embodiments, the present invention provides a kit comprising, consisting essentially of, or consisting of reagents and components useful, sufficient, or necessary for detection markers of AMD. In some embodiments, a kit comprises, consists essentially of, or consists of reagents and components for detection of 100 or fewer markers of AMD. In some embodiments, a kit comprises, consists essentially of, or consists of reagents and components for detection of 50 or fewer markers of AMD. In some embodiments, a kit comprises, consists essentially of, or consists of reagents and components for detection of 20 or fewer markers of AMD. In some embodiments, a kit comprises, consists essentially of, or consists of reagents and components for detection of 10 or fewer markers of AMD. In some embodiments, a kit comprises, consists essentially of, or consists of reagents and components for detection of 2 or more of rs147859257 (e.g., G allele), rs2230199 (e.g., C allele), and/or rs121913059 (e.g., T allele). In some embodiments, a kit comprises, consists essentially of, or consists of reagents and components for detection of 4 or more AMD biomarkers including 1, 2, or 3 of rs147859257 (e.g., G allele), rs2230199 (e.g., C allele), and/or rs121913059 (e.g., T allele). In some embodiments, a kit comprises, consists essentially of, or consists of reagents and components for detection of 6 or more AMD biomarkers including 1, 2, or 3 of rs147859257 (e.g., G allele), rs2230199 (e.g., C allele), and/or rs121913059 (e.g., T allele)In some embodiments, a kit comprises, consists essentially of, or consists of reagents and components for detection of 8 or more AMD biomarkers including 1, 2, or 3 of rs147859257 (e.g., G allele), rs2230199 (e.g., C allele), and/or rs121913059 (e.g., T allele). In some embodiments, a kit further comprises an algorithm (e.g., software, device, computer, etc.) for processing biomarker data into a risk predictive format (e.g., risk profile, risk score, etc.).

In some embodiments, the present invention provides methods for characterizing a human subject as having an increased risk for developing age-related macular degeneration (AMD), the method comprising: detecting in a sample obtained from the subject the presence of at least one G allele of the rs147859257 single nucleotide polymorphism; identifying the human subject as having wherein the presence of at least one G allele of the rs147859257 single nucleotide polymorphism is indicative of an increased risk for developing AMD. In some embodiments, characterizing a human subject as having an increased risk for developing AMD further comprises detecting in the sample obtained from the subject the presence of one or more of: at least one C allele of the rs2230199 single nucleotide polymorphism, and at least one T allele of the rs121913059 single nucleotide polymorphism; wherein the presence of at least one C allele of the rs2230199 single nucleotide polymorphism and/or at least one T allele of the rs121913059 single nucleotide polymorphism is further indicative of an increased risk for developing AMD. In some embodiments, characterizing a human subject as having an increased risk for developing AMD comprises detecting in the sample obtained from the subject the presence of each of: at least one G allele of the rs147859257, at least one C allele of the rs2230199, and at least one T allele of the rs121913059; wherein the presence of each of at least one G allele of the rs147859257, at least one C allele of the rs2230199, and at least one T allele of the rs121913059 indicates an increased risk for developing AMD. In some embodiments, characterizing a human subject as having an increased risk for developing AMD further comprises detecting in the sample obtained from the subject the presence of one or more of: at least one A allele of the rs10737680 single nucleotide polymorphism, at least one G allele of the rs3793917 single nucleotide polymorphism, at least one G allele of the rs429608 single nucleotide polymorphism, at least one C allele of the rs2230199 single nucleotide polymorphism, at least one T allele of the rs2285714 single nucleotide polymorphism, at least one T allele of the rs1329424 single nucleotide polymorphism, at least one A allele of the rs9380272 single nucleotide polymorphism, and at least one C allele of the rs493258 single nucleotide polymorphism, at least one A allele of rs1280514, at least one C allele of rs3766405, at least one C allele of rs11582939, at least one G allele of rs1048663, at least one C allele of rs412852, at least one C allele of rs11582939, and at least one G allele of rs1048663.

In some embodiments, the present invention provides methods for characterizing a human subject as having an increased risk for developing age-related macular degeneration (AMD), the method comprising: (a) detecting in a sample obtained from the subject the presence of at least one G allele of the rs147859257 single nucleotide polymorphism; and (b) identifying the human subject as having an increased risk for developing AMD based on the presence of at least one G allele of rs147859257. In some embodiments, characterizing a human subject as having an increased risk for developing AMD further comprises detecting in the sample obtained from the subject the presence of one or more of: at least one C allele of the rs2230199 single nucleotide polymorphism, and at least one T allele of the rs121913059 single nucleotide polymorphism; wherein the presence of at least one C allele of the rs2230199 single nucleotide polymorphism and/or at least one T allele of the rs121913059 single nucleotide polymorphism is further indicative of an increased risk for developing AMD. In some embodiments, characterizing a human subject as having an increased risk for developing AMD comprises detecting in the sample obtained from the subject the presence of each of: at least one G allele of the rs147859257, at least one C allele of the rs2230199, and at least one T allele of the rs121913059; wherein the presence of each of at least one G allele of the rs147859257, at least one C allele of the rs2230199, and at least one T allele of the rs121913059 indicates an increased risk for developing AMD. In some embodiments, characterizing a human subject as having an increased risk for developing AMD further comprises detecting in the sample obtained from the subject the presence of one or more of: at least one A allele of the rs10737680 single nucleotide polymorphism, at least one G allele of the rs3793917 single nucleotide polymorphism, at least one G allele of the rs429608 single nucleotide polymorphism, at least one C allele of the rs2230199 single nucleotide polymorphism, at least one T allele of the rs2285714 single nucleotide polymorphism, at least one T allele of the rs1329424 single nucleotide polymorphism, at least one A allele of the rs9380272 single nucleotide polymorphism, and at least one C allele of the rs493258 single nucleotide polymorphism, at least one A allele of rs1280514, at least one C allele of rs3766405, at least one C allele of rs11582939, at least one G allele of rs1048663, at least one C allele of rs412852, at least one C allele of rs11582939, and at least one G allele of rs1048663. In some embodiments, identifying the human subject as having an increased risk of AMD comprising diagnosing the human subject as having an increased risk of AMD.

In some embodiments, the present invention provides methods for identifying a human subject as having an increased risk for developing AMD comprising: (a) detecting in vitro the presence of at least one G allele of rs147859257 in a sample from the human subject, (b) detecting in vitro the presence of a second biomarker of AMD in a sample from the human subject, and (c) identifying the human subject as having an increased risk of AMD based on the presence of the G allele of rs147859257 and the presence of the second biomarker of AMD. In some embodiments, identifying the human subject as having an increased risk of AMD comprising diagnosing the human subject as having an increased risk of AMD. In some embodiments, the second biomarker of AMD is selected from: at least one C allele of rs2230199, and at least one T allele of rs121913059. In some embodiments, the second biomarker of AMD comprises at least one T allele of rs121913059. In some embodiments, methods further comprise:detecting in vitro the presence of at least one C allele of rs2230199 in a sample from the human subject, wherein the presence of at least one C allele of rs2230199 is further indicative of an increased risk of AMD. In some embodiments, methods further comprise detecting in vitro the presence of one or more of: at least one A allele of rs10737680, at least one G allele of rs3793917, at least one G allele of rs429608, at least one C allele of rs2230199, at least one T allele of rs2285714, at least one T allele of rs1329424, at least one A allele of rs9380272, and at least one C allele of rs493258, at least one A allele of rs1280514, at least one C allele of rs3766405, at least one C allele of rs11582939, at least one G allele of rs1048663, at least one C allele of rs412852, at least one C allele of rs11582939, and at least one G allele of rs 1048663. In some embodiments, the second biomarker of AMD is selected from: at least one A allele of rs10737680, at least one G allele of rs3793917, at least one G allele of rs429608, at least one C allele of rs2230199, at least one T allele of rs2285714, at least one T allele of rs1329424, at least one A allele of rs9380272, and at least one C allele of rs493258, at least one A allele of rs1280514, at least one C allele of rs3766405, at least one C allele of rs11582939, at least one G allele of rs1048663, at least one C allele of rs412852, at least one C allele of rs11582939, and at least one G allele of rs1048663.

In some embodiments, the present invention provides kits comprising of reagents for detection of 100 or fewer markers of AMD, at least one of the markers selected from the group consisting of: at least one G allele of the rs147859257, at least one C allele of the rs2230199, and at least one T allele of the rs121913059. In some embodiments, kits comprise reagents for detection of 50 or fewer markers of AMD. In some embodiments, kits comprise reagents for detection of 20 or fewer markers of AMD. In some embodiments, kits comprise reagents for detection of 8 or fewer markers of AMD. In some embodiments, reagents comprise amplification reagents, hybridization reagents, antibodies, etc.

In some embodiments, characterization of a subject's risk of developing AMD (e.g., identifying or diagnosing the subject as having an increased risk) is followed by treatment and/or preventative steps to reduce the subject's risk of developing AMD. In some embodiments, characterization of a subject's risk of developing AMD (e.g., identifying or diagnosing the subject as having an increased risk) is followed by treatment and/or preventative steps to delay the onset of AMD. In some embodiments, characterization of a subject's risk of developing AMD (e.g., identifying or diagnosing the subject as having an increased risk) is followed by other testing (e.g., for physical signs/symptoms of AMD).

In some embodiments, a plurality of AMD biomarkers are detected and/or analyzed to characterize a subject's risk of developing AMD (e.g., diagnose a subject as having an increased risk of developing AMD). In some embodiments, one or more detection assays are performed on a sample from a subject to detect the presence, absence, or level of a plurality of AMD biomarkers. In certain embodiments, the results of the detection assays (e.g., presence, absence, or level of various biomarkers) are processed by a risk determination and/or prediction algorithm. In some embodiments, the results of detection assays (e.g., presence, absence, or level of various AMD biomarkers) are entered into an algorithm via a computer, device, software, etc. and a risk profile or risk score is generated. In some embodiments, risk profile or risk score, generated from AMD biomarkers, is transmitted (e.g., to a clinician or the subject) or displayed (e.g., printed or displayed on a monitor). In some embodiments, treatment or other care decisions are made by the clinician and subject based on risk profile or risk score.

### BRIEF DESCRIPTION OF THE DRAWINGS

The foregoing summary and detailed description is better understood when read in conjunction with the accompanying drawings which are included by way of example and not by way of limitation.

FIG. 1 shows Ancestry based matching using the HGDP reference Panel. AMD samples' ancestry using their genotype data in panel A (PC1 and PC2) and in panel E (PC3 and PC4). Comparatively, the same set of samples' ancestries using off-target sequencing reads are shown in panel B (PC1 and PC2) and in panel E (PC3 and PC4). These four panels illustrate that similar ancestries can be inferred from either genotype data or target sequencing data. After matching 2,268 cases and 2,268 controls from AMD study and ESP study, the ancestries are shown in Panel C (PC1 and PC2) and Panel G (PC3 and PC4). Cases and controls are well matched in each graph. Further, the K155Q variant carriers' ancestries are shown in Panel D (PC1 and PC2) and Panel H (PC3 and PC4). Although the numbers of cases and controls are different, their ancestries are similarly gathered.

FIG. 2 shows depth distribution and sequencing alignment diagnostics. Panel A: Density plot comparing average sequencing depth at K155Q in targeted sequence data to that in the 1,148 sites examined in the comparison to ESP (histogram). The average sequencing depth at K155Q was 63.73. Panel B: Density plot comparing total sequencing depth at K155Q in ESP to that in the additional 1,148 ESP sites examined in the comparison to ESP (histogram). The average sequencing depth at K155Q was 90.53. Panel C: Density plot examining depth at K155Q in heterozygote carriers in the targeted sequencing sample. Average depth at K155Q was 64.11 for carriers. The histogram summarizes depth distribution across all genotyped sites. Panel D: Density plot examining depth at K155Q in heterozygote carriers in the ESP sample. The average depth at K155Q for carriers was 87.93. The histogram summarizes depth distribution across all genotyped sites.

FIG. 3 shows C3 variants R102G and K155Q and CFH variant R1210C are in the interaction domains the first alpha-macro-globular domain of C3b and CFH, respectively. The fragment of the crystal structure of the four Sushi domains (one not shown for clarity) of CFH in a complex with complement fragment C3b (PDB file: 2wii) was used to explore the effect of disease associated nonsynonymous changes. The CFH residues 987-1230 were used to generate the structure using the first four Sushi domains from 2wii as a structural template (cysteine residue side chains shown). The C-terminal Sushi domains were docked to the binding site in C3b. The first two alpha-macro-globulin domains of C3b, MG-1 and MG-2, are shown. The locations of mutations R102G, K155Q, and R1210C are marked.

### DEFINITIONS

As used herein, the term “subject” refers to any animal (e.g., a mammal), including, but not limited to, humans, non-human primates, rodents, and the like. Typically, the terms “subject” and “patient” are used interchangeably herein in reference to a human subject.

As used herein, the term “subject suspected of having AMD” refers to a subject that presents one or more symptoms indicative of age-related macular degeneration, has one or more risk factors for AMD, or is being screened for AMD (e.g., during a routine physical). A subject suspected of having AMD has generally not been tested for AMD, or has not had a recent test which indicated the subject suffers from AMD. However, a “subject suspected of having AMD” encompasses an individual who has received a preliminary diagnosis but for whom a confirmatory test has not been done. A “subject suspected of having AMD” is sometimes diagnosed with AMD and is sometimes found to not have AMD.

As used herein, the term “subject diagnosed with AMD” refers to a subject who has been tested and found to have AMD. AMD may be diagnosed using any suitable method, including but not limited to, the diagnostic methods of the present invention.

As used herein, the term “subject suffering from AMD” refers to a subject who has AMD and exhibits one or more symptoms thereof. A subject suffering from AMD may or may not have received a diagnosis, and may or may not be aware of the condition.

As used herein, the term “initial diagnosis” refers to a test result of initial AMD diagnosis that reveals the presence or absence or risk of AMD. An initial diagnosis does not include information about the stage or extent of AMD.

As used herein, the term “subject at risk for AMD” refers to a subject with one or more risk factors for developing AMD. Risk factors include, but are not limited to, gender, age, genetic predisposition, environmental exposure, and lifestyle.

As used herein, the term “characterizing AMD in subject” refers to the identification of one or more properties of AMD in a subject (e.g. degree, severity, advancement, etc.). AMD may be characterized by the identification of one or more markers (e.g., SNPs and/or haplotypes) of the present invention.

As used herein, the term “reagent(s) capable of specifically detecting biomarker expression” refers to reagents used to detect the expression of biomarkers (e.g., SNPs and/or haplotypes described herein). Examples of suitable reagents include but are not limited to, nucleic acid probes capable of specifically hybridizing to mRNA or cDNA, and antibodies (e.g., monoclonal antibodies).

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “providing a prognosis” refers to providing information regarding the impact of the presence of AMD (e.g., as determined by the diagnostic methods of the present invention) on a subject's future health.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “transgene” refers to a heterologous gene that is integrated into the genome of an organism (e.g., a non-human animal) and that is transmitted to progeny of the organism during sexual reproduction.

As used herein, the term “transgenic organism” refers to an organism (e.g., a non-human animal) that has a transgene integrated into its genome and that transmits the transgene to its progeny during sexual reproduction.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

The terms “test compound” and “candidate compound” refer to any chemical entity, pharmaceutical, drug, and the like that is a candidate for use to treat or prevent a disease, illness, sickness, or disorder of bodily function (e.g., AMD). Test compounds comprise both known and potential therapeutic compounds. A test compound can be determined to be therapeutic by screening using the screening methods of the present invention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include saliva, tissues, lacrimal fluid, and blood products, such as plasma, serum and the like. Environmental samples include environmental material such as surface matter, soil, water, and industrial samples. Such examples are not however to be construed as limiting the sample types applicable to the present invention.

### DETAILED DESCRIPTION OF EMBODIMENTS

The present invention relates generally to biomarkers for macular degeneration. In particular, the present invention provides a plurality of biomarkers (e.g., polymorphisms and/or haplotypes) for monitoring and diagnosing macular degeneration. The compositions and methods of the present invention find use in diagnostic, therapeutic, research, and drug screening applications. The present invention further provides assays for identifying, characterizing, and testing therapeutic agents that find use in treating macular degeneration.

Macular degeneration is a common cause of blindness in the elderly. To identify rare coding variants associated with a large increase in risk of age-related macular degeneration (AMD), Experiments were conducted during development of embodiments of the present invention to sequence 2,335 cases and 789 controls in 10 candidate loci (57 genes). To increase power, the control set was augmented with ancestry-matched exome sequenced controls. An analysis of coding variation in 2,268 AMD cases and 2,268 ancestry matched controls revealed two large-effect rare variants; previously described R1210C in the CFH gene (fcase=0.51%, fcontrol=0.02%, OR=23.11), and newly identified K155Q in the C3 gene (fcase=1.06%, fcontrol=0.39%, OR=2.68). The variants indicate decreased inhibition of C3 by Factor H, resulting in increased activation of the alternative complement pathway, as a key component of disease biology.

Accordingly, in some embodiments, the present invention provides methods for detection of AMD, characterizing the severity and/or advancement of AMD, and/or diagnosing a subject's susceptibility for AMD. In some embodiments, the present invention detects the presence of one or more of the SNPs described herein. The present invention is not limited by the method utilized for detection. Indeed, a variety of different methods are known to those of skill in the art including, but not limited to, microarray detection, TAQMAN, PCR, allele specific PCR, sequencing, and other methods.

In some embodiments, the present invention provides indicators (e.g. alleles, loci, SNPs, halotypes, etc.) of increased susceptibility to AMD for an individual or population. In some embodiments, a single indicator (e.g. SNP) indicates an increased AMD-susceptibility. In some embodiments, a combination of any of the SNPs listed herein indicates heightened risk of AMD or developing AMD (e.g. 2 or more SNPs, 3 or more SNPs, 4 or more SNPs, 5 or more SNPs, 6 or more SNPs, 7 or more SNPs, 8 or more SNPs, 9 or more SNPs, etc.). In some embodiments, combinations of the SNPs listed herein indicates heightened risk of AMD or developing AMD. In some embodiments, specific combinations of the SNPs listed herein indicate increased risk of AMD or developing AMD. In some embodiments, combinations of SNPs listed herein indicate increased severity of AMD. In some embodiments, increased number of SNPs listed herein indicates increased severity of AMD (e.g. 2 or more SNPs, 3 or more SNPs, 4 or more SNPs, 5 or more SNPs, 6 or more SNPs, 7 or more SNPs, 8 or more SNPs, 9 or more SNPs, etc.). In some embodiments, specific combinations of SNPs listed herein indicate increased severity of AMD. In some embodiments, a greater number of SNPs which are indicative of AMD correlates to a greater risk of AMD for an individual or population. In some embodiments, combinations of the SNPs listed herein indicate protective effects (e.g. reduced severity of AMD, reduced risk of AMD, reduced cholesterol, etc.).

In some embodiments, the compositions, methods, or kits utilize markers, listed herein, alone in combination with each other or other markers of AMD. The following references provide additional markers of AMD which may find utility in embodiments of the present invention: Kanda et al. Proc Natl Acad Sci USA. 2007 Oct. 23; 104(43):16725-6., Edwards et al. N Engl J Med. 2009 May 21; 360(21):2254-5., Fisher et al. (2005) Hum Mol Genet 14:2257-2264., Swaroop et al. (2007) Hum Mol Genet 16 Spec No. 2:R174-182., Edwards A O, et al. (2005) Complement factor H polymorphism and age-related macular degeneration. Science 308:421-424., Hageman et al. (2005) Proc Natl Acad Sci USA 102:7227-7232., Haines et al. (2005) Science 308:419-421., Klein et al. (2005) Science 308:385-389., Zareparsi et al. (2005) Am J Hum Genet 77:149-153., Clark et al. (2006) J Biol Chem 281:24713-24720., Hollyfield et al. (2008) Nat Med 14:194-198., Jakobsdottir et al. (2005) Am J Hum Genet 77:389-407., Rivera et al. (2005) Hum Mol Genet 14:3227-3236., Maller et al. (2006) Nat Genet 38:1055-1059., Schmidt et al. (2006) Am J Hum Genet 78:852-864., Kanda et al. (2007) Proc Natl Acad Sci USA 104:16227-16232., Fritsche et al. (2008) Age-related macular degeneration is associated with an unstable ARMS2 (LOC387715) mRNA. Nat Genet., U.S. Pat. No. 8,119,348, U.S. Pat. No. 7,351,524, U.S. Pat. No. 7,344,846, U.S. Pat. No. 7,108,982, U.S. Pat. No. 7,011,952, US Pub. App. No. 20090124542, US Pub. App. No. 20080318264, US Pub. App. No. 20120135884, US Pub. App. No. 20110014716, US Pub. App. No. 20090203001, US Pub. App. No. 20080280825, US Pub. App. No.20080274453, US Pub. App. No. 20080261211, US Pub. App. No. 20080146501, US Pub. App. No. 20080131418, US Pub. App. No. 20070020647, US Pub. App. No. 20060263819, US Pub. App. No.20050287601, US Pub. App. No. 20030017501, US Pub. App. No. 20020160954, US Pub. App. No.20020102581, US Pub. App. No. 20020015957, U.S. Pat. No. 7,351,534, U.S. Pat. No. 7,309,487, U.S. Pat. No. 6,593,104, and U.S. Pat. No. 6,417,342 (herein incorporated by reference in their entireties).

In some embodiments, the present invention provides kits for the detection and characterization of AMD. In some embodiments, the kits contain reagents for detecting SNPs described herein and/or antibodies specific for AMD biomarkers, in addition to detection reagents and buffers. In other embodiments, the kits contain reagents specific for the detection of AMD biomarker mRNA, SNPs, cDNA (e.g., oligonucleotide probes or primers), etc. In preferred embodiments, the kits contain all of the components necessary to perform a detection assay, including all controls, directions for performing assays, and any necessary software for analysis and presentation of results. In some embodiments, kits comprise instructions (e.g. written, digital, and/or online) to perform assays for the detection and characterization of AMD.

In some embodiments, the expression of mRNA and/or proteins associated with SNPs of the present invention are determined. In some embodiments, the presence or absence of SNPs are correlated with mRNA and/or protein expression. In some embodiments, gene silencing (e.g., siRNA and/or RNAi) is utilized to alter expression of genes associated with SNPs described herein.

In some embodiments, the present invention contemplates screening arrays of compounds (e.g., pharmaceuticals, drugs, peptides, or other test compounds) for their ability to alter expression, activity, structure, and/or interaction with other proteins, to compensate for altered function of the genes and loci disclosed herein. In some embodiments, compounds (e.g., pharmaceuticals, drugs, peptides, or other test compounds) identified using screening assays of the present invention find use in the diagnosis or treatment of AMD.

In some embodiments, the present invention provides screening assays for assessing cellular behavior or function. For example, the response of cells, tissues, or organisms to interventions (e.g., drugs, diets, aging, etc.) may be monitored by assessing, for example, cellular functions using animal or cell culture models as describe herein. Such assays find particular use for characterizing, identifying, validating, selecting, optimizing, or monitoring the effects of agents (e.g., small molecule-, peptide-, antibody-, nucleic acid-based drugs, etc.) that find use in treating or preventing AMD or related diseases or conditions.

In some embodiments, the present invention provides methods for detection of expression of AMD markers (e.g., rs147859257 (e.g., G allele), rs2230199 (e.g., C allele), and/or rs121913059 (e.g., T allele), C3 variant K155Q (or a variant comprising the K155Q mutation), C3 variant R102G (or a variant comprising the R102G mutation), and/or CFH variant R1210C (or a variant comprising the R1210C mutation), etc.). In preferred embodiments, expression is measured directly (e.g., at the RNA or protein level). In some embodiments, expression is detected in vivo or in vitro. In some embodiments, expression is detected in tissue samples (e.g., biopsy tissue). In other embodiments, expression is detected in bodily fluids (e.g., including but not limited to, plasma, serum, whole blood, mucus, and urine). In some embodiments, the present invention provides methods of identifying or characterizing AMD, or response thereof to therapy, based on the level expression of markers listed herein (e.g., mRNA or transcript levels).

The present invention further provides panels and kits for the detection of markers. In preferred embodiments, the presence of AMD marker is used to provide a prognosis to a subject. The information provided is also used to direct the course of treatment. For example, if a subject is found to have a plurality of markers indicative of AMD, therapy or other interventions can be started at an earlier point when it is more likely to be effective. In some embodiments, assaying the presence or absence of AMD markers is performed after diagnosis of AMD, but prior to treatment. In some embodiments, assaying the presence or absence of AMD markers is performed after treatment of AMD.

The present invention is not limited to the markers described herein. Any suitable marker that correlates with AMD or AMD onset or progression may be utilized, including but not limited to, those described in the illustrative examples below. Additional markers are also contemplated to be within the scope of the present invention. Any suitable method may be utilized to identify and characterize AMD markers suitable for use in the methods of the present invention, including but not limited to, those described herein.

In some embodiments, the present invention provides a panel for the analysis of a plurality of markers. The panel allows for the simultaneous analysis of multiple markers correlating with AMD. For example, a panel may include markers identified as correlating with severity of AMD, onset of AMD, and/or risk of AMD, in a subject that is/are likely or not likely to respond to a given treatment. Depending on the subject, panels may be analyzed alone or in combination in order to provide the best possible diagnosis and prognosis. Markers for inclusion on a panel are selected by screening for their predictive value using any suitable method, including but not limited to, those described herein.

In some embodiments, AMD markers are detected by measuring the expression of corresponding mRNA in a tissue or other sample (e.g., a blood sample). mRNA expression may be measured by any suitable method, including but not limited to, those disclosed below.

DNA or RNA markers may be detected, for example, by hybridization to an oligonucleotide probe. A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

In other embodiments, gene expression of AMD disease markers is detected by measuring the expression of the corresponding protein or polypeptide. Protein expression may be detected by any suitable method. In some embodiments, proteins are detected by their binding to an antibody raised against the protein.

In some embodiments, a plurality of AMD biomarkers are detected and/or analyzed to characterize a subject's risk of developing AMD (e.g., diagnose a subject as having an increased risk of developing AMD). In some embodiments, one or more detection assays are performed on a sample from a subject to detect the presence, absence, or level of a plurality of AMD biomarkers. In certain embodiments, the results of the detection assays (e.g., presence, absence, or level of various biomarkers) are processed by a risk determination and/or prediction algorithm. In some embodiments, the results of detection assays (e.g., presence, absence, or level of various AMD biomarkers) are entered into an algorithm via a computer, device, software, etc. and a risk profile or risk score is generated. In some embodiments, risk profile or risk score, generated from AMD biomarkers, is transmitted (e.g., to a clinician or the subject) or displayed (e.g., printed or displayed on a monitor). In some embodiments, treatment or other care decisions are made by the clinician and subject based on risk profile or risk score.

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of AMD to respond to a specific therapy) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or severity of disease.

In some embodiments, the present invention provides kits for the detection and characterization of AMD. In some embodiments, the kits contain reagents specific for an AMD marker, in addition to detection reagents and buffers. In other embodiments, the kits contain reagents specific for the detection of DNA or RNA (e.g., oligonucleotide probes or primers). For example, in some embodiments, the kits contain primers and reagents needed to perform PCR for detection and characterization of AMD. In some embodiments, the kits contain all of the components necessary to perform a detection assay, including all controls, directions for performing assays, and any necessary software for analysis and presentation of results.

In some embodiments, the present invention provides drug screening assays (e.g., to screen for drugs useful in treating AMD). The screening methods of the present invention utilize AMD markers identified using the methods of the present invention. For example, in some embodiments, the present invention provides methods of screening for compound that alter (e.g., increase or decrease) the expression of AMD marker genes. In some embodiments, candidate compounds are antisense agents (e.g., oligonucleotides) directed against AMD markers.

In some embodiments, alleles in linkage disequilibrium with AMD-associated SNPs affect expression or activity of downstream genes. In some embodiments, correction of these gene activities by increasing or decreasing the expression by gene-based vectors, RNAi, etc. is used for designing therapies.

This invention further pertains to novel agents identified by the screening assays described herein or other screening assays. Accordingly, it is within the scope of this invention to further use an agent identified as described herein (e.g., an AMD marker modulating agent, an antisense AMD marker nucleic acid molecule, a siRNA molecule, an AMD marker specific antibody, or an aAMD marker-binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

In some embodiments, the present invention provides therapies for AMD. In some embodiments, therapies target AMD markers (e.g., rs147859257 (e.g., G allele), rs2230199 (e.g., C allele), and/or rs121913059 (e.g., T allele), C3 variant K155Q (or a variant comprising the K155Q mutation), C3 variant R102G (or a variant comprising the R102G mutation), and/or CFH variant R1210C (or a variant comprising the R1210C mutation), etc.).

### Experimental

To systematically identify rare, large-effect variants, targeted sequencing was conducted of eight AMD risk loci identified in GWAS19 (near CFH, ARMS2, C3, C2/CFB, CFI, CETP, LIPC and TIMP3/SYN3) and two candidate regions (LPL and ABCA1) (Table 1).

TABLE 1
Target information

| | | | | Target Information | | | | | | | |
|---|---|---|---|---|---|---|---|---|---|---|---|
| | Interval | | | Protein | | | | Protein | | | |
| | Start | | | Coding | # | | | Coding | % Protein | | # Genes to |
| Chr | Position | End Position | Length | Bases | Probes | # Bases | % Interval | Bases | Coding Bases | Locus Name | region |
| 1 | 196,341,101 | 196,994,612 | 653,511 | 11,359 | 1,520 | 226,684 | 34.69 | 11,007 | 96.90 | CFH | 7 |
| 4 | 110,547,457 | 110,733,347 | 185,890 | 4,116 | 891 | 132,950 | 71.52 | 4,087 | 99.30 | CFI | 4 |
| 6 | 31,720,915 | 32,087,186 | 366,271 | 66,023 | 1,393 | 207,700 | 56.71 | 63,090 | 95.56 | C2/CFB | 29 |
| 8 | 19,786,532 | 19,938,633 | 152,101 | 1,428 | 737 | 109,963 | 72.30 | 1,418 | 99.30 | LPL | 1 |
| 9 | 107,533,234 | 107,700,286 | 167,052 | 10,408 | 860 | 128,141 | 76.71 | 10,341 | 99.36 | ABCA1 | 3 |
| 10 | 124,113,939 | 124,412,943 | 299,004 | 10,432 | 388 | 57,812 | 19.33 | 10,146 | 97.26 | ARMS2 | 5 |
| 15 | 58,555,986 | 58,870,773 | 314,787 | 1,500 | 197 | 29,453 | 9.36 | 1,488 | 99.20 | LIPC | 1 |
| 16 | 56,980,401 | 57,026,900 | 46,499 | 1,482 | 61 | 9,089 | 19.55 | 1,451 | 97.91 | CETP | 2 |
| 19 | 6,669,795 | 6,734,343 | 64,548 | 6,469 | 122 | 18,178 | 28.16 | 6,204 | 95.90 | C3 | 3 |
| 22 | 32,904,490 | 33,412,741 | 508,251 | 2,379 | 313 | 46,637 | 9.18 | 2,360 | 99.20 | SYN3/TIMP3 | 2 |
| Total | | | 2,757,914 | 115,596 | 6,482 | 966,607 | 35.05 | 111,592 | 96.54 | | 57 |

These regions were re-sequenced in 3,124 individuals (2,335 cases and 789 controls) recruited in ophthalmology clinics at the University of Michigan and at the University of Pennsylvania and among Age-Related Eye Disease Study (AREDS) participants (Chen et al. Proc Natl Acad Sci USA 107, 7401-6 (2010); Age-Related Eye Disease Study Research. Ophthalmology 107, 2224-32 (2000); herein incorporated by reference in their entireties). Genomic targets were enriched using a set of 150-bp probes designed by Agilent Technologies, and sequence data was generated on Illumina Genome Analyzer and HiSeq instruments. The ten loci comprised 115,596 nucleotides of protein coding sequence and totaled 2,757,914 nucleotides overall. Probes were designed to capture 111,592 nucleotides (96.5% of coding sequence) and 966,607 nucleotides overall (35.1% of the locus sequence), generating an average of 123,221,974 mapped bases of on-target sequence per individual (an 127.5x average depth counting bases with quality >20 in reads with mapping quality >30, after duplicate read removal); 98.49% of sites with designed probes were covered at >10× depth. The same variant calling tools and quality control filters similar were applied to those used to analyze NHLBI Exome Sequencing Project data21 (Table 2).

TABLE 2
Summary of analyzed variants.

| | | |
|---|---|---|
| Initial Call Set | Protein Coding Regions | Sites Compared To ESP |

Target Summary

| | | | |
|---|---|---|---|
| Targeted nucleotides | 2,757,914 | 115,596 | — |
| Examined nucleotides | 966,607 | 111,592 | 97,196 |
| Mean coverage | 106.8 | 128.6 | 133.0 |
| Fraction >10×^{#} | .95 (.92-.99) | .98 (.98-1.00) | .98 (.98-1.00) |

Overall
SNP

| | | | |
|---|---|---|---|
| No. sites | 31,527 | 2,368 | 1,148 |
| No. in 1000 Genomes Phase I | 11,721 | 750 | 707 |
| No. of dbSNP 135 | 12,571 | 1,017 | 797 |
| Fraction Novel* | 59.82% | 55.03% | 25.78% |
| No. synonymous | 834 | 834 | 280 |
| No. nonsynonymous | 1,380 | 1,380 | 416 |
| No. nonsense | 43 | 43 | 10 |
| Ts/Tv ratio | 2.09 | 2.88 | 2.73 |

Variation Per Sample
SNP

| | | | |
|---|---|---|---|
| No. sites | 1,714 | 78 | 89 |
| No. in 1000 Genomes Phase I | 1,650 | 75 | 88 |
| No. of dbSNP 135 | 1,691 | 76 | 87 |
| Fraction Novel* | 1% | 0% | 0% |
| No. synonymous | 40 | 40 | 24 |
| No. nonsynonymous | 34 | 34 | 19 |
| No. nonsense | 1 | 1 | 1 |

^{#}Fraction of variant sites covered. We showed average values and quartile ranges are shown within parentheses.
*Fraction novel denotes the fractions of variants that not reported in 1000 Genomes Project Phase 1 or dbSNP 135.

An average of 1,714 non-reference sites were identified in each sequenced individual. In total, this resulted in 31,527 single nucleotide variants of which 18,956 were not in dbSNP 135. Discovered sites included 834 synonymous variants, 1,380 nonsynonymous variants and 43 nonsense variants. Among 13 samples sequenced in duplicate, genotype concordance was 99.82% (when depth >10x). Among 908 samples previously examined with GWAS arrays19, sequence-based genotypes were 98.99% concordant with arraybased calls (again, when depth >10×).

In an initial comparison of AMD cases and controls (see Table 3), no rare coding variants with frequency <1% reached experiment wide significance, although several showed encouraging patterns.

TABLE 3
Initial statistical association analysis of AMD 2,335 cases and 789 controls.

| | | | | | | Frequency | | | | |
|---|---|---|---|---|---|---|---|---|---|---|
| | | | Nearest | | Alleles | (alt allele) | | | | Conditional |
| SNP | Chromosome | Position (bp) | Gene | Consequence | (ref/alt) | Cases | Controls | OR | P-value | P-value |

Common variant hits

| | | | | | | | | | |
|---|---|---|---|---|---|---|---|---|---|
| rs1061170 | 1 | 196,659,237 | CFH | H402Y | C/T | 0.481 | 0.662 | 0.47 | 4.48 × 10⁻³⁶ |
| rs641153 | 6 | 31,914,180 | C2 | R32Q | G/A | 0.060 | 0.105 | 0.55 | 1.26 × 10⁻⁸ |
| rs10490924 | 10 | 124,214,448 | ARMS2 | A69S | G/T | 0.326 | 0.184 | 2.15 | 1.85 × 10⁻²⁸ |
| rs2230199 | 19 | 6,718,387 | C3 | R102G | G/C | 0.247 | 0.175 | 1.55 | 2.31 × 10⁻⁹ |

Rare variant hits with MAF <1% and P < .01 (after conditioning on nearby common variants).

| | | | | | | | | | | |
|---|---|---|---|---|---|---|---|---|---|---|
| rs121913059 | 1 | 196,716,375 | CFH | R1210C | C/T | 0.005 | 0.000 | ∞ | 2.57 × 10⁻³ | 2.00 × 10⁻⁴ |
| rs143667999 | 6 | 31,922,453 | RDBP | D208E | G/C | 0.001 | 0.005 | 0.21 | 5.99 × 10⁻³ | 6.70 × 10⁻³ |
| rs147859257 | 19 | 6,718,146 | C3 | K155Q | T/G | 0.010 | 0.003 | 3.27 | 6.30 × 10⁻³ | 2.50 × 10⁻³ |

For example, rare variant R1210C in the CFH gene was observed in 23 of the 2,335 sequenced cases, but in none of the 789 sequenced controls. Common variants in several loci exhibited strong evidence of association, including in CFH (peak variant rs9427642 with case frequency fcase=12%, control frequency fcontrol=27%, P-value=2.52×10−48), ARMS2 (rs10490924, fcase=33%, fcontrol=18%, P-value=5.48×10−27), C3 (rs2230199, fcase=25%, fcontrol=17%, P-value=3.94×10−9), C2/CFB (rs556679, fcase=7%, fcontrol=12%, P-value=1.32×10−10).

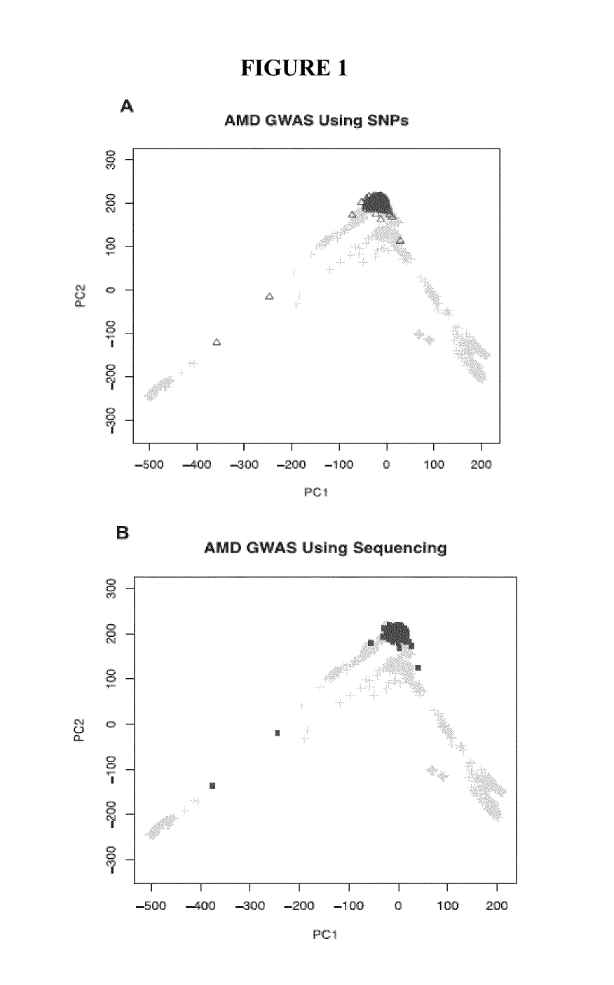

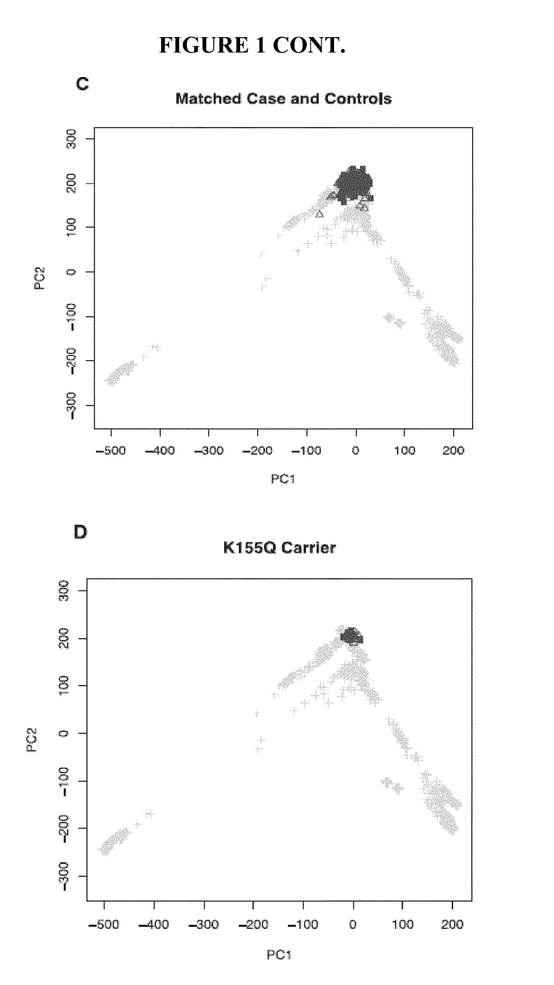

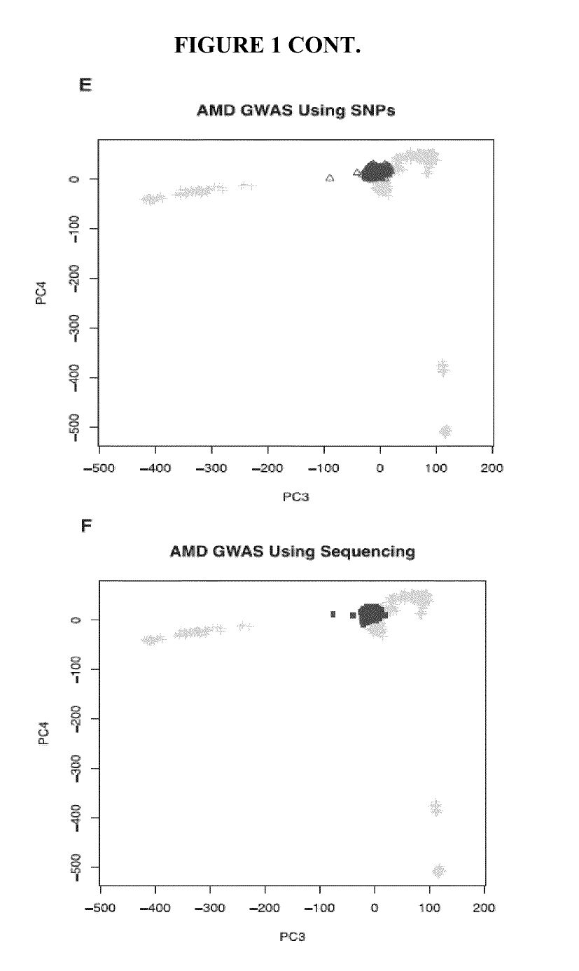

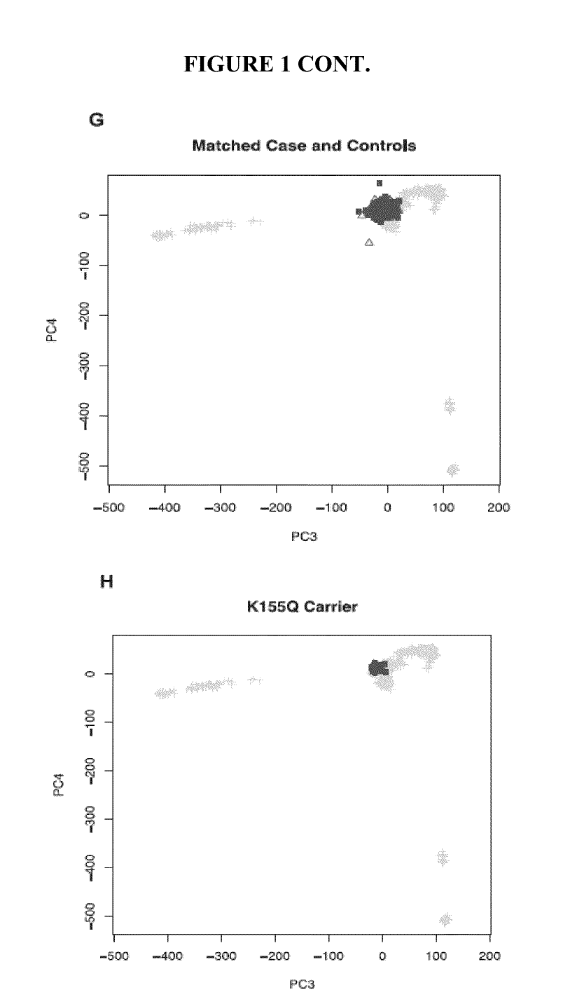

A key requirement for establishing significance of rare disease associated variants is the availability of sufficient numbers of control samples. To increase power, steps were taken to identify additional controls, focusing on samples from the NHLBI Exome Sequencing Project (ESP), which sequenced 15,336 genes across 6,515 individuals (Fu et al. Nature (2012); herein incorporated by reference in its entirety). Sequence data for test samples and the NHLBI Exome Sequencing Project samples were analyzed with the same analysis pipeline, which minimized potential differences due to heterogeneity in analysis tools and parameters. To further avoid sequencing and variant calling artifacts, analysis was restricted to sites within regions targeted in both sequencing experiments, genotyped and covered with >10 reads in >90% of the samples examined in each project, and >5-bp away from insertion/deletion polymorphisms catalogued by the 1000 Genomes Project (The 1000 Genomes Project Consortium et al. Nature 491, 56-65 (2012); herein incorporated by reference in its entirety). An ancestry-matched subset of our samples and of samples from the NHLBI Exome Sequencing Project (The 1000 Genomes Project Consortium et al. Nature 491, 56-65 (2012); Mathieson & McVean. Nat Genet 44, 243-6 (2012); herein incorporated by reference in their entireties). Principal component analysis was used to construct a genetic ancestry map of the world with samples from the Human Genome Diversity Project, each genotyped at 632,958 SNPs25,26. If GWAS array genotypes were available for test samples and for the NHLBI Exome Sequencing Project samples, it would be straightforward to place them directly in this genetic ancestry map. Using targeted sequence data, however, the analysis is more challenging: targeted regions include too few variants to accurately represent global ancestry and off-target regions are covered too poorly, precluding estimation of the accurate genotypes needed for standard principal component analysis. Thus, a new algorithm was developed (Wang et al. Nat Genet (2013); herein incorporated by reference in its entirety) to place each sequenced sample in a pre-defined genetic ancestry map of the world. The method can accurately place individuals on this worldwide ancestry map with <0.05× average coverage of the genome and is thus ideal for targeted sequence data, such as ours and the NHLBI Exome Sequence data, which have average off-target coverage of ˜0.23× and 0.90×, respectively (See FIGS. 1A, 1B, 1E and 1F), which shows that PCA coordinates inferred using 0.10× genome coverage or using GWAS array genotypes are highly similar). The focus was on samples where PCA coordinates could be estimated confidently (Procrustes similarity larger than 0.95; see Supplementary Methods) and a greedy algorithm was used to match cases and controls based on estimated genetic ancestry. Alternative matching algorithms do not alter the conclusions. After matching, a set of 2,268 AMD cases and 2,268 ancestry-matched controls, matched one-to-one was used (See FIGS. 1C and 1G). Since AMD phenotype information was not available for most controls, we expect that a small proportion may eventually develop disease; however, this will not impact power substantially (Wellcome Trust Case Control Consortium. Nature 447, 661-78 (2007); herein incorporated by reference in its entirety). After matching case-control samples, 1 variant was excluded with Hardy-Weinberg Equilibrium test, and the focus was placed on the 430 remaining nonsynonymous variants.

In this expanded analysis (see Table 4), common variant signals at all loci increased in significance (in comparison to Table 3).

TABLE 4
Summary association ressults.

| | | | | | | Frequency | | | | |
|---|---|---|---|---|---|---|---|---|---|---|
| | | | Nearest | | Alleles | (alt allele) | | | | Conditional |
| SNP | Chromosome | Position(bp) | Gene | Consequence | (ref/alt) | Cases | Controls | OR | P-value | P-value |

Common variant hits

| | | | | | | | | | |
|---|---|---|---|---|---|---|---|---|---|
| rs200244837 | 1 | 196,884,290 | CFH | Intron:CFHR4 | T/A | 0.020 | 0.109 | 0.17 | 6.7 × 10⁻⁷³ |
| rs6467 | 6 | 32,006,858 | C2 | Intron:CYP21A2 | C/A | 0.760 | 0.637 | 1.81 | 1.2 × 10⁻³⁷ |
| rs255 | 8 | 19,811,901 | LPL | Intron:LPL | T/C | 0.151 | 0.088 | 1.83 | 3.6 × 10⁻²⁰ |
| rs45519541 | 10 | 124,183,691 | ARMS2 | Intron:PLEKHA1 | T/C | 0.145 | 0.037 | 4.41 | 1.5 × 10⁻⁷⁵ |
| rs11076176 | 16 | 57,007,446 | CETP | Intron:CETP | T/G | 0.145 | 0.177 | 0.79 | 4.4 × 10⁻⁵ |
| rs2230199 | 19 | 6,718,387 | C3 | R102G | G/C | 0.253 | 0.206 | 1.30 | 1.8 × 10⁻⁷ |

Rare variant hits MAF <1% Conditional P < .01 Sort by P

| | | | | | | | | | | |
|---|---|---|---|---|---|---|---|---|---|---|
| rs121913059 | 1 | 196,716,375 | CFH | R1210C | C/T | 0.005 | 0.000 | 23.11 | 2.9 × 10⁻⁶ | 6.0 × 10⁻⁴ |
| rs147859257 | 19 | 6,718,146 | C3 | K155Q | T/G | 0.011 | 0.004 | 2.68 | 2.7 × 10⁻⁴ | 2.8 × 10⁻⁵ |

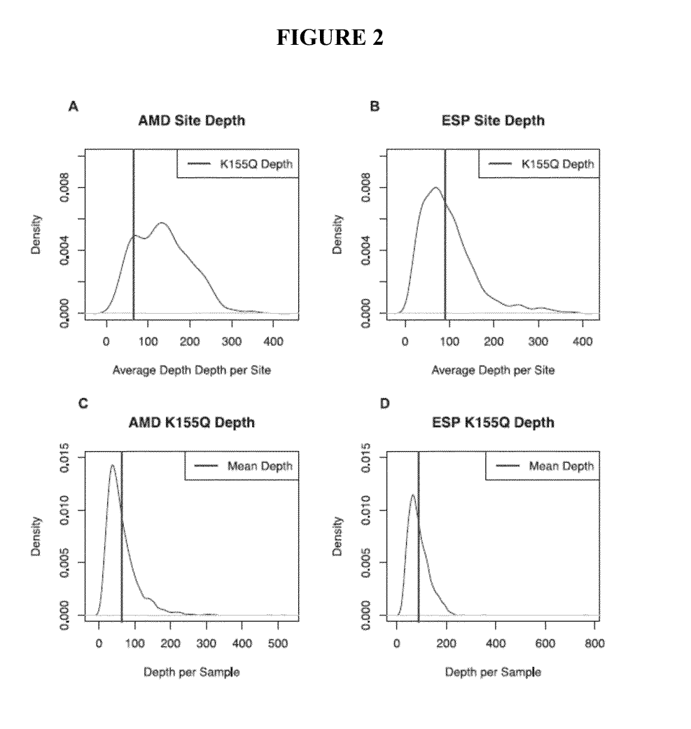

In addition, two rare coding variants exhibited association. The first was R1210C in the CFH gene (observed in one control and 23 cases, OR=23.11, pexact=2.9×10−6), providing strong support for the original report15. The second variant was K155Q in the C3 gene (18 controls, 48 cases, OR=2.68, pexact=2.7×10−4; See FIG. 1D and 1G for carrier ancestry distribution). When controlling for a previously described common variant signal nearby, rs2230199 (fcontrol=20.63%, fcase=25.26%, marginal pexact=1.8×10−7, OR=1.31), the evidence for association with K155Q increased slightly (conditional OR=2.91, pexact=2.8×10−5). Inspection of the raw read data shows the variant is well supported and is unlikely to be a sequencing or alignment artifact (See FIG. 2). Further strong evidence for association of this variant with macular degeneration is provided in independent work by deCODE Genetics, examining 1,143 Icelandic macular degeneration cases and 51,435 Icelandic controls (control frequency 0.55%, OR=3.45, p=1.1×10−7; Helgason et al. A rare nonsynonymous sequence variant in C3 confers high risk of age-related macular degeneration. Nat Genet (2013).). In 1,606 directly genotyped cases of macular degeneration from the Age Related Disease Study 1129 the variant has frequency 1.77%, similar to our sequenced AMD cases (frequency 1.10%) and the deCODE AMD cases and is notably higher than in our sequenced controls (0.30%), in NHLBI Exome Sequencing Project participants with primarily European Ancestry (0.40%) and in deCODE controls (0.55%).

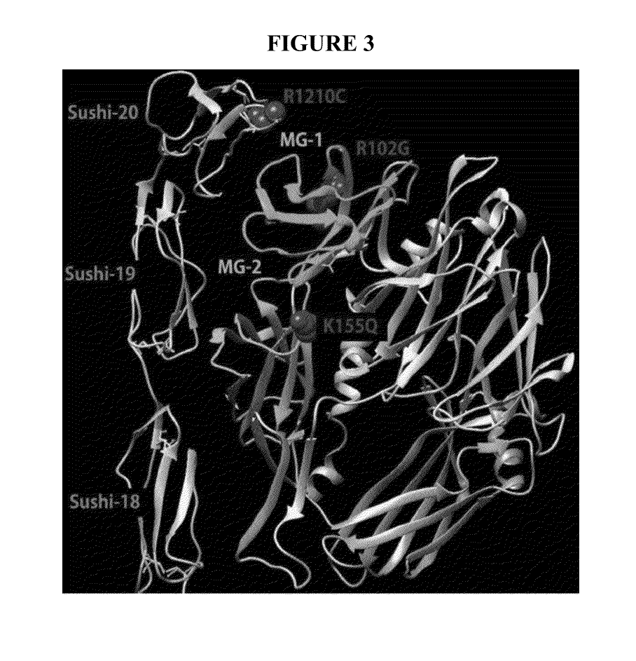

The functional consequences of the K155Q variant was then investigated. FIG. 3 shows that CFH variant R1210C (OR=23.11), C3 variant K155Q (OR=2.91) and C3 variant R102G (OR=1.31) all map near the surface where CFH and C3b interact and can potentially affect binding of complement factor H to C3b. Factor H inhibits C3b and limits immune responses mediated by the alternate component pathway. The analysis of crystal structures summarized in FIG. 4 indicates that K155Q and R102G can affect binding of the first macro-globular domain of C3 to CFH and thus potentially interfere with inactivation of the alternative component pathway30. The three variants (R102G and K155Q in C3 and R1210C in CFH) all are associated with replacement of a positively charged residue.

All publications and patents mentioned in the present application and/or listed below are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

### REFERENCES

1. Priya, R. R., Chew, E. Y. & Swaroop, A. Genetic Studies of Age-related Macular Degeneration: Lessons, Challenges, and Opportunities for Disease Management. Ophthalmology 119, 2526-36 (2012).
2. Swaroop, A., Chew, E. Y., Rickman, C. B. & Abecasis, G. R. Unraveling a multifactorial late-onset disease: from genetic susceptibility to disease mechanisms for age-related macular degeneration. Annu Rev Genomics Hum Genet 10, 19-43 (2009).
3. Friedman, D. S. et al. Prevalence of age-related macular degeneration in the United States. Archives of Ophthalmology 122, 564-72 (2004).
4. Haines, J. L. et al. Complement factor H variant increases the risk of age-related macular degeneration. Science 308, 419-21 (2005).
5. Edwards, A. O. et al. Complement factor H polymorphism and age-related macular degeneration. Science 308, 421-4 (2005).
6. Klein, R. J. et al. Complement factor H polymorphism in age-related macular degeneration. Science 308, 385-9 (2005).
7. Jakobsdottir, J. et al. Susceptibility genes for age-related maculopathy on chromosome 10q26. Am J Hum Genet 77, 389-407 (2005).
8. Yates, J. R. et al. Complement C3 variant and the risk of age-related macular degeneration. N Engl J Med 357, 553-61 (2007).
9. Gold, B. et al. Variation in factor B (BF) and complement component 2 (C2) genes is associated with age-related macular degeneration. Nat Genet 38, 458-62 (2006).
10. Fagerness, J. A. et al. Variation near complement factor I is associated with risk of advanced AMD. Eur J Hum Genet 17, 100-4 (2009).
11. Maller, J. B. et al. Variation in complement factor 3 is associated with risk of age-related macular degeneration. Nat Genet 39, 1200-1 (2007).
12. Fritsche, L. G. et al. Seven New Loci Associated with Age-Related Macular Degeneration. Nature Genetics (in press)(2013).
13. Arakawa, S. et al. Genome-wide association study identifies two susceptibility loci for exudative agerelated macular degeneration in the Japanese population. Nat Genet 43, 1001-4 (2011).
14. Nejentsev, S., Walker, N., Riches, D., Egholm, M. & Todd, J. A. Rare variants of IFIH1, a gene implicated in antiviral responses, protect against type 1 diabetes. Science 324, 387-9 (2009).
15. Raychaudhuri, S. et al. A rare penetrant mutation in CFH confers high risk of age-related macular degeneration. Nature Genetics 43, 1232-6 (2011).
16. Jozsi, M. et al. Factor H and atypical hemolytic uremic syndrome: mutations in the C-terminus cause structural changes and defective recognition functions. J Am Soc Nephrol 17, 170-7 (2006).
17. Manuelian, T. et al. Mutations in factor H reduce binding affinity to C3b and heparin and surface attachment to endothelial cells in hemolytic uremic syndrome. J Clin Invest 111, 1181-90 (2003).
18. Ferreira, V. P. et al. The binding of factor H to a complex of physiological polyanions and C3b on cells is impaired in atypical hemolytic uremic syndrome. J Immunol 182, 7009-18 (2009).
19. Chen, W. et al. Genetic variants near TIMP3 and high-density lipoprotein-associated loci influence susceptibility to age-related macular degeneration. Proc Natl Acad Sci USA 107, 7401-6 (2010).
20. Age-Related Eye Disease Study Research, G. Risk factors associated with age-related macular degeneration. A case-control study in the age-related eye disease study: Age-Related Eye Disease Study Report Number 3. Ophthalmology 107, 2224-32 (2000).
21. Tennessen, J. A. et al. Evolution and functional impact of rare coding variation from deep sequencing of human exomes. Science 337, 64-9 (2012).
22. Fu, W. et al. Analysis of 6,515 exomes reveals the recent origin of most human protein-coding variants. Nature (2012).
23. The 1000 Genomes Project Consortium et al. An integrated map of genetic variation from 1,092 human genomes. Nature 491, 56-65 (2012).
24. Mathieson, I. & McVean, G. Differential confounding of rare and common variants in spatially structured populations. Nat Genet 44, 243-6 (2012).
25. Li, J. Z. et al. Worldwide human relationships inferred from genome-wide patterns of variation. Science 319, 1100-4 (2008).
26. Wang, C. et al. Estimating Individual Ancestry Using Next Generation Sequencing. Nat Genet (2013).
27. Wellcome Trust Case Control Consortium. Genome-wide association study of 14,000 cases of seven common diseases and 3,000 shared controls. Nature 447, 661-78 (2007).
28. Helgason, H. et al. A rare nonsynonymous sequence variant in C3 confers high risk of age-related macular degeneration. Nat Genet (2013).
29. Group, A. R. et al. The Age-Related Eye Disease Study 2 (AREDS2): study design and baseline characteristics (AREDS2 report number 1). Ophthalmology 119, 2282-9 (2012).
30. Heurich, M. et al. Common polymorphisms in C3, factor B, and factor H collaborate to determine systemic complement activity and disease risk. Proc Natl Acad Sci USA 108, 8761-6 (2011).

We claim:

1. A method for characterizing a human subject as having an increased risk for developing age-related macular degeneration (AMD), said method comprising: detecting in a sample obtained from said subject the presence of at least one G allele of the rs147859257 single nucleotide polymorphism; identifying said human subject as having wherein the presence of at least one G allele of the rs147859257 single nucleotide polymorphism is indicative of an increased risk for developing AMD.

2. The method of claim 1, wherein characterizing a human subject as having an increased risk for developing AMD further comprises detecting in said sample obtained from said subject the presence of one or more of: at least one C allele of the rs2230199 single nucleotide polymorphism, and at least one T allele of the rs121913059 single nucleotide polymorphism; wherein the presence of at least one C allele of the rs2230199 single nucleotide polymorphism and/or at least one T allele of the rs121913059 single nucleotide polymorphism is further indicative of an increased risk for developing AMD.

3. The method of claim 1, wherein characterizing a human subject as having an increased risk for developing AMD comprises detecting in said sample obtained from said subject the presence of each of: at least one G allele of the rs147859257, at least one C allele of the rs2230199, and at least one T allele of the rs121913059; wherein the presence of each of at least one G allele of the rs147859257, at least one C allele of the rs2230199, and at least one T allele of the rs121913059 indicates an increased risk for developing AMD.

4. The method of claim 1, wherein characterizing a human subject as having an increased risk for developing AMD further comprises detecting in said sample obtained from said subject the presence of one or more of: at least one A allele of the rs10737680 single nucleotide polymorphism, at least one G allele of the rs3793917 single nucleotide polymorphism, at least one G allele of the rs429608 single nucleotide polymorphism, at least one C allele of the rs2230199 single nucleotide polymorphism, at least one T allele of the rs2285714 single nucleotide polymorphism, at least one T allele of the rs1329424 single nucleotide polymorphism, at least one A allele of the rs9380272 single nucleotide polymorphism, and at least one C allele of the rs493258 single nucleotide polymorphism, at least one A allele of rs1280514, at least one C allele of rs3766405, at least one C allele of rs11582939, at least one G allele of rs1048663, at least one C allele of rs412852, at least one C allele of rs11582939, and at least one G allele of rs1048663.

5. A method for characterizing a human subject as having an increased risk for developing age-related macular degeneration (AMD), said method comprising:
(a) detecting in a sample obtained from said subject the presence of at least one G allele of the rs147859257 single nucleotide polymorphism; and
(b) identifying said human subject as having an increased risk for developing AMD based on the presence of at least one G allele of rs147859257.

6. The method of claim 5, wherein characterizing a human subject as having an increased risk for developing AMD further comprises detecting in said sample obtained from said subject the presence of one or more of: at least one C allele of the rs2230199 single nucleotide polymorphism, and at least one T allele of the rs121913059 single nucleotide polymorphism; wherein the presence of at least one C allele of the rs2230199 single nucleotide polymorphism and/or at least one T allele of the rs121913059 single nucleotide polymorphism is further indicative of an increased risk for developing AMD.

7. The method of claim 5, wherein characterizing a human subject as having an increased risk for developing AMD comprises detecting in said sample obtained from said subject the presence of each of: at least one G allele of the rs147859257, at least one C allele of the rs2230199, and at least one T allele of the rs121913059; wherein the presence of each of at least one G allele of the rs147859257, at least one C allele of the rs2230199, and at least one T allele of the rs121913059 indicates an increased risk for developing AMD.

8. The method of claim 5, wherein characterizing a human subject as having an increased risk for developing AMD further comprises detecting in said sample obtained from said subject the presence of one or more of: at least one A allele of the rs10737680 single nucleotide polymorphism, at least one G allele of the rs3793917 single nucleotide polymorphism, at least one G allele of the rs429608 single nucleotide polymorphism, at least one C allele of the rs2230199 single nucleotide polymorphism, at least one T allele of the rs2285714 single nucleotide polymorphism, at least one T allele of the rs1329424 single nucleotide polymorphism, at least one A allele of the rs9380272 single nucleotide polymorphism, and at least one C allele of the rs493258 single nucleotide polymorphism, at least one A allele of rs1280514, at least one C allele of rs3766405, at least one C allele of rs11582939, at least one G allele of rs1048663, at least one C allele of rs412852, at least one C allele of rs11582939, and at least one G allele of rs1048663.

9. The method of claim 5, wherein identifying said human subject as having an increased risk of AMD comprising diagnosing said human subject as having an increased risk of AMD

10. The method of claim 5, further comprising detecting in vitro the presence of a second biomarker of AMD in a sample from said human subject, and identifying said human subject as having an increased risk of AMD based on the presence of said G allele of rs147859257 and the presence of said second biomarker of AMD.

11. The method of claim 10, wherein identifying said human subject as having an increased risk of AMD comprising diagnosing said human subject as having an increased risk of AMD

12. The method of claim 10 wherein said second biomarker of AMD is selected from: at least one C allele of rs2230199, and at least one T allele of rs121913059.

13. The method of claim 12 wherein said second biomarker of AMD comprises at least one T allele of rs121913059.

14. The method of claim 13, further comprising: detecting in vitro the presence of at least one C allele of rs2230199 in a sample from said human subject, wherein the presence of at least one C allele of rs2230199 is further indicative of an increased risk of AMD.

15. The method of claim 13, further comprising: detecting in vitro the presence of one or more of: at least one A allele of rs10737680, at least one G allele of rs3793917, at least one G allele of rs429608, at least one C allele of rs2230199, at least one T allele of rs2285714, at least one T allele of rs1329424, at least one A allele of rs9380272, and at least one C allele of rs493258, at least one A allele of rs1280514, at least one C allele of rs3766405, at least one C allele of rs11582939, at least one G allele of rs1048663, at least one C allele of rs412852, at least one C allele of rs11582939, and at least one G allele of rs1048663.

16. The method of claim 10 wherein said second biomarker of AMD is selected from: at least one A allele of rs10737680, at least one G allele of rs3793917, at least one G allele of rs429608, at least one C allele of rs2230199, at least one T allele of rs2285714, at least one T allele of rs1329424, at least one A allele of rs9380272, and at least one C allele of rs493258, at least one A allele of rs1280514, at least one C allele of rs3766405, at least one C allele of rs11582939, at least one G allele of rs1048663, at least one C allele of rs412852, at least one C allele of rs11582939, and at least one G allele of rs1048663.

17. A kit comprising of reagents for detection of 100 or fewer markers of AMD, at least one of said markers selected from the group consisting of: at least one G allele of the rs147859257, at least one C allele of the rs2230199, and at least one T allele of the rs121913059.

18. The kit of claim 17 comprising of reagents for detection of 50 or fewer markers of AMD.

19. The kit of claim 18 comprising of reagents for detection of 20 or fewer markers of AMD.

20. The kit of claim 19 comprising of reagents for detection of 8 or fewer markers of AMD.
